(19) United States
(12) Patent Application Publication
(10) Pub. No.: US 2024/0130979 A1
(43) Pub. Date: Apr. 25, 2024

(54) COMPOSITIONS AND METHODS FOR TREATMENT OF SKIN CONDITIONS

(71) Applicant: EpiTop, Inc., Poway, CA (US)

(72) Inventors: Byung Eui KIM, Greenwood Village, CO (US); Donald Y. M. LEUNG, Denver, CO (US)

(21) Appl. No.: 18/046,125

(22) Filed: Oct. 12, 2022

(51) Int. Cl. A61K 31/015; A61K 8/31; A61P 17/00; A61P 17/02; A61Q 19/08

(52) U.S. Cl. CPC A61K 31/015; A61K 8/31; A61P 17/00; A61P 17/02; A61Q 19/08

(57) ABSTRACT

Provided are a composition and a method for preventing, treating, or ameliorating a skin disease or condition. In some cases, the method comprises administering to the subject in need thereof a therapeutically effective amount of a composition comprising at least about 10 v/v % of thujopsene. In some cases, the skin disease or condition comprises a skin inflammatory disease, an allergic disease, a skin wound, a skin aging-related condition, or particulate matter (PM_{2.5})-associated condition.

### BACKGROUND

Various skin barrier defects are common characteristics of inflammatory skin diseases, allergic diseases, skin wounds, skin aging-related conditions, or particulate matter (PM_{2.5})-associated conditions. Recently these skin diseases or disorders became common health problems. Despite continuous efforts to develop the treatments of the skin diseases and disorders, there remains an unmet need for approaches that overcome inherent limitations of conventional methods to effectively treat them.

### SUMMARY

In one aspect, the disclosure provides a method for preventing, treating, or ameliorating a skin disease or condition in a subject in need thereof. In some embodiments, the method comprises administering to the subject a therapeutically effective amount of a composition comprising at least about 10 v/v % of thujopsene. In some embodiments, the composition comprises at least about 30 v/v % of thujopsene. In some embodiments, the composition comprises at least about 50 v/v % of thujopsene. In some embodiments, the composition comprises at least about 70 v/v % of thujopsene. In some embodiments, the composition comprises at least about 90 v/v % of thujopsene. In some embodiments, the thujopsene has a purity of at least about 80%. In some embodiments, the thujopsene has a purity of at least about 90%. In some embodiments, the thujopsene has a purity of at least about 95%. In some embodiments, the purity of thujopsene is determined by HPLC.

In some embodiments, the skin disease or condition comprises a skin inflammatory disease, an allergic disease, a skin wound, a skin aging-related condition, or particulate matter (PM_{2.5})-associated condition. In some embodiments, the skin inflammatory disease comprises atopic dermatitis (AD), contact dermatitis, seborrheic dermatitis, acne, psoriasis (PS), xeroderma, UV-induced inflammation, ichthyosis, or combinations thereof. In some embodiments, the allergic disease comprises anaphylaxis, allergic rhinitis, asthma, atopic dermatitis, food allergies, urticaria, eczema, or combinations thereof. In some embodiments, the skin wound comprises chronic wound, puncture wound, surgical wound, thermal wound, chemical wound, burn, abrasion, avulsion, laceration, or combinations thereof. In some embodiments, the skin aging-related condition comprises wrinkling, sagging, thinning, the appearance of age spots, dryness, or combinations thereof. In some embodiments, the PM_{2.5}-associated condition comprises environmental allergic diseases. In some embodiments, the composition is configured to increase the expression of one or more genes selected from filaggrin (FLG), loricrin (LOR), sirtuin 1 (SIRT1), beta-defensin-3 (EMD-3), cathelicidin (LL-37), and Olfactory Receptor Family 2 Subfamily AT Member 4 (OR2AT4). In some embodiments, the composition is configured to decrease or inhibit the activity of one or more cytokines selected from interleukin (IL)-4, IL-6, IL-13, IL-25, IL-31, and IL-33. In some embodiments, the one or more cytokines is selected from IL-4, IL-6, and IL-13.

In some embodiments, the therapeutically effective amount of the composition is administered one to five times a day. In some embodiments, the therapeutically effective amount of the composition is administered two times a day. In some embodiments, the therapeutically effective amount of the composition is administered three times a day. In some embodiments, the therapeutically effective amount of the composition is administered for at least five consecutive days. In some embodiments, the therapeutically effective amount of the composition is administered for at least seven consecutive days. In some embodiments, the therapeutically effective amount of the composition is administered at least for about 15 days, about 21 days, about 24 days, about 28 days, or about 30 days.

In some embodiments, the therapeutically effective amount of the composition comprises from about 0.001%, about 0.005%, about 0.010%, about 0.015%, about 0.020%, about 0.025%, about 0.030%, about 0.035%, about 0.040%, about 0.045%, about 0.050%, about 0.055%, about 0.060%, about 0.065%, about 0.070%, about 0.075%, about 0.080%, about 0.085%, about 0.090%, about 0.095%, or about 0.1% of concentration. In some embodiments, the therapeutically effective amount of the composition comprises from about 0.1%, about 0.2%, about 0.3%, about 0.4%, about 0.5%, about 0.6%, about 0.7%, about 0.8%, about 0.9%, or about 1.0% of concentration. In some embodiments, the therapeutically effective amount of the composition comprises from about 1%, about 2%, about 3%, about 4%, about 5%, about 6%, about 7%, about 8%, about 9%, or about 10% of concentration. In some embodiments, the therapeutically effective amount of the composition comprises from about 10%, about 20%, about 30%, about 40%, or about 50% of concentration.

In some embodiments, the administering comprises topical administration or transdermal administration. In some embodiments, the topical administration comprises administration of the composition to an affected skin area. In some embodiments, the topical administration comprises administering an ointment, cream, suspensions, paste, lotions, powders, solutions, oils, encapsulated gel, liposomes, sprayable aerosol or vapors, or any combination thereof.

In one aspects, the disclosure provides a pharmaceutical composition for preventing, treating, or ameliorating a skin disease or condition in a subject in need thereof. In some embodiments, the composition comprises at least 10 v/v % of thujopsene. In some embodiments, the composition comprises at least about 30 v/v % of thujopsene. In some embodiments, the composition comprises at least about 50 v/v % of thujopsene. In some embodiments, the composition comprises at least about 70 v/v % of thujopsene. In some embodiments, the composition comprises at least about 90 v/v % of thujopsene. In some embodiments, the thujopsene has a purity of at least about 80%. In some embodiments, the thujopsene has a purity of at least about 90%. In some embodiments, the thujopsene has a purity of at least about 95%. In some embodiments, the purity of the thujopsene is determined by HPLC.

In some embodiments, the composition optionally further comprises at least one pharmaceutically acceptable carrier, at least one diluent, at least one excipient or at least one additive. In some embodiments, the at least one pharmaceutically acceptable carrier is selected from the group consisting of: dimethyl sulfoxide (DMSO), alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal, and combinations thereof. In some embodiments, the at least one diluent comprises buffer, saline, water, DMSO, lactose, or combinations thereof. In some embodiments, the at least one excipient is selected from the group consisting of: animal and vegetable fats, oils, waxes, paraffins, starch, tragacanth, cellulose derivatives, polyethylene glycols, silicones, bentonites, silicic acid, talc, zinc oxide, lactose, talc, silicic acid, aluminum hydroxide, calcium silicates, polyamide powder, and combinations thereof. In some embodiments, the at least one additive is selected from the group consisting of: a fatty substance, an organic solvent, a solubilizing agent, a thickener, a gelling agent, a softener, an antioxidant, a suspending agent, a stabilizer, a foaming agent, an aromatic, a surfactant, water, an ionic or non-ionic emulsifying agent, a filler, a sequestering agent, a chelating agent, a preservative, vitamins, a blocker, a moisturizing agent, essential oil, a dye, a pigment, a hydrophilic or hydrophobic activator, a lipid vesicle, antiseptics, stabilizing agents, hydrating agents, emulsification promoters or salts and/or buffers for osmotic control, and combinations thereof.

In some embodiments, the skin disease or condition comprises a skin inflammatory disease, an allergic disease, a skin wound, a skin aging-related condition, or PM_{2.5}-associated condition. In some embodiments, the skin inflammatory disease comprises atopic dermatitis (AD), contact dermatitis, seborrheic dermatitis, acne, psoriasis (PS), xeroderma, UV-induced inflammation, ichthyosis, or combinations thereof. In some embodiments, the allergic disease comprises anaphylaxis, allergic rhinitis, asthma, atopic dermatitis, food allergies, urticaria, eczema, or combinations thereof. In some embodiments, the skin wound comprises chronic wound, puncture wound, surgical wound, thermal wound, chemical wound, burn, abrasion, avulsion, laceration, or combinations thereof. In some embodiments, the skin aging-related condition comprises wrinkling, sagging, thinning, the appearance of age spots, dryness, or combinations thereof. In some embodiments, the PM_{2.5}-associated condition comprises environmental allergic diseases. In some embodiments, the composition is configured to increase the expression of one or more genes selected from FLG, LOR, SIRT1, RBD-3, OR2AT2, and LL-37. In some embodiments, the composition is configured to decrease or inhibit the activity of one or more cytokines selected from IL-4, IL-6, IL-13, IL-25, IL-31, and IL-33. In some embodiments, the one or more cytokines is selected from IL-4, IL-6, and IL-13.

In another aspects, the disclosure provides a composition for use in a method for preventing, treating, or ameliorating a skin disease or condition in a subject in need thereof. In some embodiments, the method comprises administering to the subject a therapeutically effective amount of a composition comprising at least about 10 v/v % of thujopsene. In some embodiments, the composition comprises at least about 30 v/v % of thujopsene. In some embodiments, the composition comprises at least about 50 v/v % of thujopsene. In some embodiments, the composition comprises at least about 70 v/v % of thujopsene. In some embodiments, the composition comprises at least about 90 v/v % of thujopsene. In some embodiments, the thujopsene has a purity of at least about 80%. In some embodiments, the thujopsene has a purity of at least about 90%. In some embodiments, the thujopsene has a purity of at least about 95%. In some embodiments, the purity of thujopsene is determined by HPLC.

In some embodiments, the composition optionally further comprises at least one pharmaceutically acceptable carrier, at least one diluent, at least one excipient or at least one additive. In some embodiments, the at least one pharmaceutically acceptable carrier is selected from the group consisting of: dimethyl sulfoxide (DMSO), alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal, and combinations thereof. In some embodiments, the at least one diluent comprises buffer, saline, water, DMSO, lactose, or combinations thereof. In some embodiments, the at least one excipient is selected from the group consisting of: animal and vegetable fats, oils, waxes, paraffins, starch, tragacanth, cellulose derivatives, polyethylene glycols, silicones, bentonites, silicic acid, talc, zinc oxide, lactose, talc, silicic acid, aluminum hydroxide, calcium silicates, polyamide powder, and combinations thereof. In some embodiments, the at least one additive is selected from the group consisting of: a fatty substance, an organic solvent, a solubilizing agent, a thickener, a gelling agent, a softener, an antioxidant, a suspending agent, a stabilizer, a foaming agent, an aromatic, a surfactant, water, an ionic or non-ionic emulsifying agent, a filler, a sequestering agent, a chelating agent, a preservative, vitamins, a blocker, a moisturizing agent, essential oil, a dye, a pigment, a hydrophilic or hydrophobic activator, a lipid vesicle, antiseptics, stabilizing agents, hydrating agents, emulsification promoters or salts and/or buffers for osmotic control, and combinations thereof.

In some embodiments, the skin disease or condition comprises a skin inflammatory disease, an allergic disease, a skin wound, a skin aging-related condition, or PM_{2.5}-associated condition. In some embodiments, the skin inflammatory disease comprises atopic dermatitis (AD), contact dermatitis, seborrheic dermatitis, acne, psoriasis (PS), xeroderma, UV-induced inflammation, ichthyosis, or combinations thereof. In some embodiments, the allergic disease comprises anaphylaxis, allergic rhinitis, asthma, atopic dermatitis, food allergies, urticaria, eczema, or combinations thereof. In some embodiments, the skin wound comprises chronic wound, puncture wound, surgical wound, thermal wound, chemical wound, burn, abrasion, avulsion, laceration, or combinations thereof. In some embodiments, the skin aging-related condition comprises wrinkling, sagging, thinning, the appearance of age spots, dryness, or combinations thereof. In some embodiments, the PM_{2.5}-associated condition comprises environmental allergic diseases.

In some embodiments, the composition is configured to increase the expression of one or more genes selected from FLG, LOR, SIRT1, RBD-3, OR2T2, and LL-37. In some embodiments, the composition is configured to decrease or inhibit the activity of one or more cytokines selected from IL-4, IL-6, IL-13, IL-25, IL-31, and IL-33. In some embodiments, the one or more cytokines is selected from IL-4, IL-6, and IL-13.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A displays gene expressions of FLG in human primary keratinocytes by various concentrations of thujopsene, a mixture of IL-4 and IL-13, and IFN- [symbol] , according to some embodiments of the present disclosure.

FIG. 1B displays gene expressions of LOR in human primary keratinocytes by various concentrations of thujopsene, a mixture of IL-4 and IL-13, and IFN-γ, according to some embodiments of the present disclosure.

FIG. 2 shows gene expressions of FLG in human primary keratinocytes by thujopsene, a mixture of IL-4 and IL-13, and a mixture of thujopsene, IL-4 and IL-13, according to some embodiments of the present disclosure.

FIG. 3A displays gene expressions of FLG in human primary keratinocytes from atopic dermatitis patients by thujopsene, according to some embodiments of the present disclosure.

FIG. 3B displays gene expressions of LOR in human primary keratinocytes from atopic dermatitis patients by thujopsene, according to some embodiments of the present disclosure.

FIGS. 4A and 4B display that thujopsene blocks PM_{2.5}-mediated inhibition of epidermal barrier proteins in human primary keratinocytes, according to some embodiments of the present disclosure.

FIG. 5A-D display that thujopsene improves wound healing and prevents skin cancer development by upregulation of 1-EBD-3 (5A), LL-37 (5B), OR2AT4 (5C), and SIRT1 (5D), according to some embodiments of the present disclosure.

FIG. 6A-B display gene expression of IL-6 (6A) and SIRT1 (6B) to show that thujopsene inhibits aging and wrinkling process, according to some embodiments of the present disclosure.

FIG. 7 shows that thujopsene up to 0.04 v/v % is not toxic to NHEKs.

### DETAILED DESCRIPTION

The main function of the skin is to act as a barrier that separates the internal organs from the external environment, thus protecting against environmental stress, chemical/physical damage, bacterial infection, and the like. Non-limiting examples of the functions include regulation of the transepidermal water loss and defense against the action of external physico-chemical agents and aggression of microorganisms. Therefore, dysfunction of skin barrier can enhance allergic sensitization through the skin (PMCID: PMC7291847, PMCID: PMC7494573). Epithelial barrier dysfunction can initiate the development of atopic dermatitis (AD) and allergic diseases (PMCID: PMC7291847, PMCID: PMC7494573, PMCID: PMC7676854). Epidermal barrier proteins such as filaggrin (FLG) and loricrin (LOR) are known to play pivotal roles in maintaining normal skin barrier function (PMCID: PMC6436854, PMCID: PMC5911439).

Various skin barrier defects are common characteristic findings of inflammatory skin diseases, allergic diseases, skin wounds, skin aging-related conditions, or particulate matter (PM_{2.5})-associated conditions. Recently these skin diseases or disorders became common health problems. These skin diseases or disorders have been reported to account for 1.79% of all diseases worldwide. The American Academy of Dermatology Association reports that one in four Americans suffers from a skin condition. Despite continuous efforts to develop the treatments of the skin diseases and disorders, there remain challenges to treat them effectively. New biologics have been used for patients with chronic inflammatory skin diseases such as AD and psoriasis. However, they cannot treat the skin conditions effectively and are costly. Medical costs for AD and psoriasis are about $5.3 billion and $35 billion a year, respectively, in the US. In addition, they are not allowed for children younger than 2 years old although AD prevalence is high in infants.

Particulate matter (PM) is one of the major air pollutants and a major health concern that continues to grow with industrialization and urbanization (PMCID: PMC4465283, PMID: 31554704, PMCID: PMC6528157). The impact of PM is notable, given its socioeconomic burden and the fact that about 9 million people die of PM-associated diseases per year worldwide (PMCIDs: PMC6528157, PMC6156628, PMID 32447079). PM is classified on the basis of its aerodynamic diameter: PM₁₀ (<10 μm), PM_{2.5} (<2.5 μm), and PM_{0.1} (<0.1 μm) (PMCID: PMC44652831). The majority of the particle mass is in the fraction with less than 2.5 μm, and these particles can carry a large amount of absorbed pollutants, oxidants, and organic compounds (PMID 25947313). This disclosure focuses on PM_{2.5} for the experiments and invention.

Disclosed herein are solutions to these and other problems known in the art. The present disclosure provides a pharmaceutical composition and a method to prevent, treat or ameliorate a skin disease or condition. The pharmaceutical composition disclosed herein comprises at least about 10 v/v % of thujopsene. Thujopsene is a natural chemical compound, classified as a sesquiterpene, with the molecular formula C₁₅H₂₄. In some embodiments, the pharmaceutical composition prevents, treats or ameliorates the skin diseases by enhancing and/or restoring the skin barrier function. In some embodiments, the composition disclosed herein induces/enhances expressions of filaggrin and antimicrobial peptides such as beta-defensin-3 (HBD-3) and cathelicidin (LL-37), thus enhancing skin barrier function and treating the skin disease and condition. In some embodiments, the composition disclosed herein attenuates the inhibitory effect of proinflammatory cytokines on FLG expression. In some embodiments, the composition is configured to decrease or inhibit the activity of one or more cytokines selected from interleukin (IL)-4, IL-6, IL-13, IL-25, IL-31, and IL-33. In some embodiments, the skin disease or condition comprises a skin inflammatory disease, an allergic disease, a skin wound, a skin aging-related condition, or particulate matter (PM_{2.5})-associated condition.

### Skin Barrier

The function of the skin is to provide an effective barrier between the internal organism and external environments, providing protection and support to the organism. The epidermal barrier protects against mechanical, chemical, and microbial injury through the formation of terminally differentiated keratinocytes, a process termed keratinization. During keratinization, epidermal cells progressively mature from the basal epidermal layers to form flattened cells of the stratum corneum (SC). Within the epidermis, keratinocyte proliferation is restricted to the basal cell layers. After mitosis in the basal layer, keratinocytes differentiate and migrate through the epidermis towards the SC. The differentiation process yields several keratinocyte layers within the epidermis. Distinct marker genes are expressed by keratinocytes at each of the differentiation stages. As the outermost layer of the skin, with a thickness of 10-20 μm, the SC is the primary mediator of the epidermal permeability barrier, accounting for over 90% of the functionality of the skin. The complex tissue of the SC is composed of supports execution of corneocytes and a matrix of intercellular lipids (ceramide cholesterol, and free fatty acids), with both components derived from the terminal differentiation process of keratinocytes. The bulk of the mechanical resistance by the epidermal barrier is due to corneocytes. A protein shell called the corneocyte envelope surrounds each corneocyte and its components include loricrin (LOR), involucrin, and filaggrin (FLG). Beyond the corneocyte and in immediate contact with it sits the corneocyte lipid envelope, which is a structure of specialized lipids. These lipids and protein-rich corneocytes are critical for the formation of the functional skin barrier.

Filaggrin (FLG) is a crucial component of the cornified envelope in the outer layer of the epidermis, promoting the collapse of the cell into a flattened shape, which is characteristic of corneocytes in the cornified layer (Palmer et al., Journal of Investigative Dermatology, 130(7), 1922-1930, 2006). FLG is synthesized initially as profilaggrin (pro-FLG). During cornification, pro-FLG is dephosphorylated and proteolytically cleaved to release individual FLG molecules (Resing et al., Biochemistry, 24, 4167-4175, 1985). Decreased expression of FLG in the skin and mutations with loss of function in FLG gene have been described in atopic dermatitis (AD). Sirtuin 1 (SIRT1) is a protein that contribute to autoimmune disease. The activation of SIRT1 therapeutically may trigger autoimmune disease. SIRT1 is known to be effective in wound healing (Qiang et al., Rep 2017), promote keratinocyte differentiation (Blander et al., 2009), and prevent UVR-related premature aging process and cancer development (Bielach-Bazyluk et al. cells, PMID: 33917352). SIRT1 acts as a tumor suppressor through its role in DNA damage response, genome integrity, and tumor suppression (Wang et al., Cancer cell, 2008, PMID: 18835033). SIRT1 also has anti-aging effects (PMID: 24011076). Cathelicidin (LL-37) and human beta defensin (HBD)-3 are antimicrobial peptides and important elements of the innate immune responses. HBD-3 and LL-37 induce cell proliferation and migration, and stimulate re-epithelialization and angiogenesis (Hirsch et al., 2009; Nakatsuji and Gallo 2012). HBD-3 and LL-37 also have beneficial effects on wound healing (Gibson et al., 2012; Carretero et al., 2008). Activation of Olfactory Receptor Family 2 Subfamily AT Member 4 (OR2AT4) enhances wound healing by inducing proliferation, migration, and re-epithelialization (Busse et al., 2014). Stimulation of OR2AT4 with an odorant ligand positively affected keratinocyte proliferation, migration, and regeneration (Busse et al., 2014).

Innate lymphoid cells do not express antigen-specific receptors, myeloid, or dendritic cell markers. Innate lymphoid cells are important for innate immune responses to infection and the regulation of inflammation and metabolism. Among innate lymphoid cells, Group 2 respond to local Th2 antigens derived from helminth and viral infection and produce type 2 cytokines such as interleukin (IL)-4, IL-5, IL-9, and IL-13. These lead to the allergic inflammation process. IL-4 and IL-13 downregulate the expression of skin barrier proteins, including FLG, LOR, and involucrin. Among various interleukins, IL-6 induces aging process and wrinkling (PMID: 23659624).

### Compositions

The present disclosure provides a pharmaceutical composition for preventing, treating, or ameliorating a skin disease or condition in a subject. In some embodiments, the composition comprises at least 10 v/v % of thujopsene. In some embodiments, the skin disease or condition comprises a skin inflammatory disease, a skin wound, a skin aging-related condition, or particulate matter (PM_{2.5})-associated condition. In some embodiments, the composition optionally further comprises at least one pharmaceutically acceptable carrier, at least one diluent, at least one excipient or at least one additive.

The pharmaceutical composition disclosed herein comprises about 10 v/v %, about 20 v/v %, about 30 v/v %, about 40 v/v %, about 50 v/v %, about 60 v/v %, about 70 v/v %, about 80 v/v %, about 90 v/v %, or about 100 v/v % of thujopsene. In some embodiments, the composition comprises at least about 10 v/v %, about 20 v/v %, about 30 v/v %, about 40 v/v %, about 50 v/v %, about 60 v/v %, about 70 v/v %, about 80 v/v %, about 90 v/v %, or about 100 v/v % of thujopsene. In some embodiments, the composition comprises at most about 10 v/v %, about 20 v/v %, about 30 v/v %, about 40 v/v %, about 50 v/v %, about 60 v/v %, about 70 v/v %, about 80 v/v %, about 90 v/v %, or about 100 v/v % of thujopsene. In some embodiments, the composition comprises from about 10 v/v % to about 100 v/v %, from about 20 v/v % to about 90 v/v %, from about 30 v/v % to about 80 v/v %, from about 40 v/v % to about 70 v/v %, or from about 50 v/v % to about 60 v/v % of thujopsene.

A purity of the thujopsene is determined by HPLC. In some embodiments, the thujopsene has a purity of about 80%, about 82%, about 84%, about 86%, about 88%, about 90%, about 92%, about 94%, about 95%, or about 100%. In some embodiments, the thujopsene has a purity of at least about 80%, about 82%, about 84%, about 86%, about 88%, about 90%, about 92%, about 94%, about 95%, or about 100%. In some embodiments, the thujopsene has a purity of at most about 80%, about 82%, about 84%, about 86%, about 88%, about 90%, about 92%, about 94%, about 95%, about 100%. In some embodiments, the thujopsene has a purity of from about 80% to about 100%, from about 82% to about 95%, from about 84% to about 94%, from about 86% to about 92%, or from about 88% to about 90%. In some embodiments, the thujopsene has a purity of about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

The pharmaceutical composition disclosed herein optionally further comprises at least one pharmaceutically acceptable carrier, at least one diluent, at least one excipient or at least one additive. Non-limiting examples of the at least one pharmaceutically acceptable carrier comprises dimethyl sulfoxide (DMSO), alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal, and combinations thereof. In some embodiments, concentration of the DMSO is about 0.05%, about 0.075%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 20%, about 30%, about 40%, about 50%, about 60%, about 70%, about 80%, or about 90%. In some embodiments, the concentration of DMSO is at least about 0.05%, about 0.075%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 20%, about 30%, about 40%, about 50%, about 60%, about 70%, about 80%, or about 90%. In some embodiments, the concentration of DMSO is at most about 0.05%, about 0.075%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 20%, about 30%, about 40%, about 50%, about 60%, about 70%, about 80%, or about 90%. In some embodiments, concentration of the DMSO is from about 0.05% to about 90%, from about 0.075% to about 80%, from about 0.1% to about 70%, from about 0.2% to about 60%, from about 0.3% to about 50%, from about 0.4% to about 40%, from about 0.5% to about 30%, from about 0.6% to about 20%, from about 0.7% to about 10%, from about 0.4% to about 40%, from about 0.5% to about 30%, from about 0.6% to about 20%, from about 0.7% to about 10%, from about 0.8% to about 9%, from about 0.9% to about 8%, from about 1% to about 7%, from about 2% to about 6%, from about 3% to about 5%, or from about 4% to about 90%. In some embodiments, the at least one diluent comprises gentamicin/amphotericin. In some embodiments, concentration of the gentamicin/amphotericin is about 0.2%, about 0.4%, about 0.6%, about 0.8%, about 1%, about 1.2%, about 1.4%, about 1.6%, about 1.8%, about 2%, about 3%, about 4%, about 5%, about 6%, about 7%, about 8%, about 9%, about 10%, about 20%, about 30%, about 40%, about 50%, about 60%, about 70%, about 80%, or about 90%. In some embodiments, concentration of the gentamicin/amphotericin is at least about 0.2%, about 0.4%, about 0.6%, about 0.8%, about 1%, about 1.2%, about 1.4%, about 1.6%, about 1.8%, about 2%, about 3%, about 4%, about 5%, about 6%, about 7%, about 8%, about 9%, about 10%, about 20%, about 30%, about 40%, about 50%, about 60%, about 70%, about 80%, or about 90%. In some embodiments, concentration of the gentamicin/amphotericin is at most about 0.2%, about 0.4%, about 0.6%, about 0.8%, about 1%, about 1.2%, about 1.4%, about 1.6%, about 1.8%, about 2%, about 3%, about 4%, about 5%, about 6%, about 7%, about 8%, about 9%, about 10%, about 20%, about 30%, about 40%, about 50%, about 60%, about 70%, about 80%, or about 90%. In some embodiments, concentration of the gentamicin/amphotericin is from about 0.2% to about 90%, from about 0.3% to about 80%, from about 0.4% to about 70%, from about 0.5% to about 60%, from about 0.6% to about 50%, from about 0.7% to about 40%, from about 0.4% to about 30%, from about 0.5% to about 20%, from about 0.6% to about 10%, from about 0.7% to about 9%, from about 0.8% to about 8%, from about 0.9% to about 7%, from about 1% to about 6%, from about 2% to about 5%, or from about 3% to about 4%. Non-limiting examples of the at least one diluent are buffer, saline, water, DMSO, lactose, or combinations thereof.

The pharmaceutical composition disclosed herein optionally further comprises at least one pharmaceutically acceptable excipient. In some embodiments, the at least one excipient is selected from the group consisting of: animal and vegetable fats, oils, waxes, paraffins, starch, tragacanth, cellulose derivatives, polyethylene glycols, silicones, bentonites, silicic acid, talc, zinc oxide, lactose, talc, silicic acid, aluminum hydroxide, calcium silicates, polyamide powder, and combinations thereof. In some embodiments, the at least one excipient is naturally occurring. In another embodiments, at least one excipient is non-naturally occurring.

The pharmaceutical composition disclosed herein optionally further comprises at least one pharmaceutically acceptable additives. Non-limiting examples of the at least one additive comprise a fatty substance, an organic solvent, a solubilizing agent, a thickener, a gelling agent, a softener, an antioxidant, a suspending agent, a stabilizer, a foaming agent, an aromatic, a surfactant, water, an ionic or non-ionic emulsifying agent, a filler, a sequestering agent, a chelating agent, a preservative, vitamins, a blocker, a moisturizing agent, essential oil, a dye, a pigment, a hydrophilic or hydrophobic activator, a lipid vesicle, antiseptics, stabilizing agents, hydrating agents, emulsification promoters or salts and/or buffers for osmotic control, and combinations thereof. In some embodiments, the at least one additive is naturally occurring. In another embodiments, at least one additive is non-naturally occurring. In some embodiments, the at least one additive comprises other therapeutically useful substances. In additional embodiments, the at least one additive further comprises absorption enhancers, permeation enhancers, thickening agents, viscosity enhancers, agents for adjusting and/or maintaining the pH, agents to adjust the osmotic pressure, preservatives, surfactants, buffers, salts (preferably sodium chloride), suspending agents, dispersing agents, solubilizing agents, stabilizers and/or tonicity agents.

### Skin Diseases or Conditions

The present disclosure provides a pharmaceutical composition and a method for preventing, treating or ameliorating a skin disease or condition in a subject. In some embodiments, the skin disease or condition comprises a skin inflammatory disease, an allergic disease, skin wound, a skin aging-related condition, or particulate matter (PM_{2.5})-associated condition.

In some embodiments, the skin inflammatory disease comprises atopic dermatitis (AD), contact dermatitis, seborrheic dermatitis, acne, psoriasis (PS), xeroderma, UV-induced inflammation, ichthyosis, or combinations thereof. AD is a skin disorder associated with extensive barrier dysfunction and elevated interleukin (IL)-4 and IL-13 signatures. The barrier dysfunction can be induced by the deficiency of filaggrin (FLG), loricrin (LOR), and involucrin (IVL) because these are major epidermal barrier proteins to maintain normal skin barrier function. IL-4 and IL-13 inhibit the expression of FLG, LOR, and IVL by activating signal transducer and activator of transcription (STAT)6 and STAT3. Contact dermatitis is a skin response with an itchy rash to direct contact with a substance or an allergic reaction to it. In some cases, the expression of FLG and LOR is downregulated in patients with allergic contact dermatitis. Seborrheic dermatitis is a common chronic inflammatory skin disorder that mainly affects your scalp causing scaly patches, red skin and stubborn dandruff. Seborrheic dermatitis is related to the expression of many genes important in epidermal barrier formation such as FLG and LOR. Acne is the most common skin disorder encountered in ambulatory dermatology practice in the United States that predominantly affects teenagers, but can also affect preadolescents and post-teen individuals. In some cases, acne may be associated with inherent abnormalities in epidermal barrier functions. Psoriasis is an immune-mediated disease that causes inflammation in the body, affecting more than 3% of the US adult population (more than 7.5 million US adults). In some cases, it causes a rash with itchy, scaly patches, most commonly on the knees, elbows, trunk and scalp. Psoriasis skin shows deficiency of FLG and LOR and has skin barrier defects consequently (PMCID: PMC8609659, PMC4436234). In some cases, variant in FLG gene may be associated with psoriasis. Xeroderma refers to a common dry skin condition which results in skin roughness, tightness, flaking, and scaling. In some cases, xeroderma is associated with FLG mutations. In other cases, xeroderma is associated with allergic contact dermatitis. In some cases, UV irradiation may elicit inflammation in the skin by increasing proinflammatory cytokine production in keratinocytes. In some cases, FLG genotype may in part determine the photoprotective capacity of human skin. Ichthyosis is a skin condition where the skin becomes dry, thick, and scaly caused by loss-of-function mutations in FLG gene. The present disclosure provides a pharmaceutical composition and a method for preventing, treating or ameliorating the skin inflammatory diseases.

In recent years, the overall prevalence of allergic diseases in childhood and adolescence have increased. The pharmaceutical composition disclosed herein provides treatment or ameliorate the allergic disease. In some embodiments, the allergic disease comprises anaphylaxis, allergic rhinitis, asthma, atopic dermatitis, food allergies, urticaria, eczema, or combinations thereof. In some cases, people carrying FLG mutations may have a high-risk to develop severe food allergy reactions, such as anaphylaxis. Anaphylaxis is a serious, life-threatening allergic reaction to foods, insect stings, medications, and latex. In some cases, anaphylaxis causes itchiness, redness, or just a mild warming of the skin. In some cases, FLG mutations may be a significant risk factor for later childhood asthma, eczema, or rhinitis. In some cases, asthma, eczema, or rhinitis may make your skin dry, rash, red, or itchy.

In some embodiments, the skin wound comprises chronic wound, puncture wound, surgical wound, thermal wound, chemical wound, burn, abrasion, avulsion, laceration, or combinations thereof. When the skin is wounded, the wound site is filled with blood and adjacent skin resident cells by natural healing action that results in a wound healing process taking place. Induction of keratinocyte proliferation at the wound edge is one of the initial events during cutaneous wound healing that critically contributes to the efficiency of wound closure.

Additionally, the compositions disclosed herein can be effective for use in the skin aging-related condition. In some embodiments, the skin aging-related condition comprises wrinkling, sagging, thinning, the appearance of age spots, dryness, or combinations thereof. In some cases, the composition may improve skin anti-aging, reducing skin wrinkles, skin elasticity, skin moisturizing, skin dryness, or skin regeneration.

The pharmaceutical composition and method disclosed herein relate to the particulate matter (PM_{2.5})-associated condition. In some embodiments, the PM_{2.5}-associated condition comprises environmental allergic diseases. PM is one of the major air pollutants and a major health concern that continue to grow these days. About 9 million people die of PM-associated diseases per year worldwide. PM is classified based on its aerodynamic diameter. PM_{2.5} represents aerodynamic diameter less than 2.5 μm. A large number of absorbed pollutants, oxidants, and organic compounds can be carried by PM_{2.5}. It has been reported that PM penetrates the epidermis through hair follicles in normal, intact skin, and causes cutaneous inflammation in a mouse model. The epidermis is the outmost part of the skin, providing a physical and functional barrier to prevent invasions of PM to the human body. Epithelial barrier dysfunction induces type 2 immune responses and is considered an initial step in developing AD. PM_{2.5} is known to inhibit filaggrin (FLG) expressions and increase transepidermal water loss. In some embodiments, the composition disclosed herein prevents, treats or ameliorates the PM_{2.5}-associated condition.

In some embodiments, the composition is configured to increase the expression of one or more genes selected from filaggrin (FLG), loricrin (LOR), sirtuin 1 (SIRT1), beta-defensin-3 (HBD-3), cathelicidin (LL-37), and Olfactory Receptor Family 2 Subfamily AT Member 4 (OR2AT4).

LOR is the most abundant component of the cornified envelope, which is an insoluble and tough structure formed beneath the cell membrane. The main function of the cornified cell envelope is to provide human skin with a protective barrier against the environment.

In some embodiments, the composition is configured to decrease or inhibit the activity of one or more cytokines selected from interleukin (IL)-4, IL-6, IL-13, IL-25, IL-31, and IL-33. In some embodiments, the one or more cytokines is selected from IL-4, IL-6, and IL-13.

### Methods

The present disclosure also provides a method of preventing, treating, or ameliorating a skin disease or condition by administering to the subject in need thereof a therapeutically effective amount of a composition comprising at least about 10 v/v % of thujopsene. In some embodiments, the composition comprises about 10 v/v %, about 20 v/v %, about 30 v/v %, about 40 v/v %, about 50 v/v %, about 60 v/v %, about 70 v/v %, about 80 v/v %, about 90 v/v %, or 100 v/v % of thujopsene. In some embodiments, the composition comprises at least about 10 v/v %, about 20 v/v %, about 30 v/v %, about 40 v/v %, about 50 v/v %, about 60 v/v %, about 70 v/v %, about 80 v/v %, about 90 v/v %, or about 100 v/v % of thujopsene. In some embodiments, the composition comprises at most about 10 v/v %, about 20 v/v %, about 30 v/v %, about 40 v/v %, about 50 v/v %, about 60 v/v %, about 70 v/v %, about 80 v/v %, about 90 v/v %, or about 100 v/v % of thujopsene. In some embodiments, the composition comprises from about 10 v/v % to about 100 v/v %, from about 20 v/v % to about 90 v/v %, from about 30 v/v % to about 80 v/v %, from about 40 v/v % to about 70 v/v %, or from about 50 v/v % to about 60 v/v % of thujopsene.

A purity of the thujopsene is determined by HPLC. In some embodiments, the thujopsene has a purity of about 80%, about 82%, about 84%, about 86%, about 88%, about 90%, about 92%, about 94%, about 95%, or about 100%. In some embodiments, the thujopsene has a purity of at least about 80%, about 82%, about 84%, about 86%, about 88%, about 90%, about 92%, about 94%, about 95%, or about 100%. In some embodiments, the thujopsene has a purity of at most about 80%, about 82%, about 84%, about 86%, about 88%, about 90%, about 92%, about 94%, about 95%, or about 100%. In some embodiments, the thujopsene has a purity of from about 80% to about 100%, from about 82% to about 95%, from about 84% to about 94%, from about 86% to about 92%, or from about 88% to about 90%.

This disclosure relates to the skin disease or condition comprising a skin inflammatory disease, an allergic disease, a skin wound, a skin aging-related condition, or particulate matter (PM_{2.5})-associated condition. In some embodiments, the skin inflammatory disease comprises atopic dermatitis (AD), contact dermatitis, seborrheic dermatitis, acne, psoriasis (PS), xeroderma, UV-induced inflammation, ichthyosis, or combinations thereof. In some embodiments, the allergic disease comprises anaphylaxis, allergic rhinitis, asthma, atopic dermatitis, food allergies, urticaria, eczema, or combinations thereof. In other embodiments, the skin wound comprises chronic wound, puncture wound, surgical wound, thermal wound, chemical wound, burn, abrasion, avulsion, laceration, or combinations thereof. In yet other embodiments, the skin aging-related condition comprises wrinkling, sagging, thinning, the appearance of age spots, dryness, or combinations thereof. In still other embodiments, the PM_{2.5}-associated condition comprises environmental allergic diseases.

In some embodiments, the composition is configured to increase the expression of one or more genes selected from FLG, LOR, SIRT1, RBD-3, LL-37, and OR2AT4. In some embodiments, the composition is configured to decrease or inhibit the activity of one or more cytokines selected from IL-4, IL-6, IL-13, IL-25, IL-31, and IL-33. In some embodiments, the one or more cytokines is selected from IL-4, IL-6, and IL-13.

In some embodiments, the therapeutically effective amount of the composition is administered one to five times a day. In some embodiments, the therapeutically effective amount of the composition is administered one, twice, three times, four times or five times a day. In some embodiments, the therapeutically effective amount of the composition is administered one to five times, twice to four times, or three times to five times a day. In some embodiments, the therapeutically effective amount of the composition is administered for at least five consecutive days. In some embodiments, the therapeutically effective amount of the composition is administered for at least six consecutive days. In some embodiments, the therapeutically effective amount of the composition is administered for at least seven consecutive days. In some embodiments, the therapeutically effective amount of the composition is administered for at least five, at least six, or at least seven consecutive days. In some embodiments, the therapeutically effective amount of the composition is administered for at most five, at least six, or at least seven consecutive days. In some embodiments, the therapeutically effective amount of the composition is administered for from five to seven consecutive days. In some embodiments, the therapeutically effective amount of the composition is administered about 15 days, about 21 days, about 24 days, about 28 days, or about 30 days. In some embodiments, the therapeutically effective amount of the composition is administered at most for about 15 days, about 21 days, about 24 days, about 28 days, or about 30 days. In some embodiments, the therapeutically effective amount of the composition is administered for from about 15 days to about 30 days, from about 21 days to about 28 days, or from about 24 days to about 30 days. In some embodiments, the therapeutically effective amount of the composition is administered for from at least about 1 week, at least about 2 weeks, at least about 4 weeks, at least about 2 months, at least about 4 months, or at least about 6 months.

In some embodiments, the therapeutically effective amount of the composition ranges from about 0.001% to about 0.04% of concentration. In some embodiments, the therapeutically effective amount of the composition comprises from about 0.005% to about 0.02% of concentration. In some embodiments, the therapeutically effective amount of the composition comprises from about 0.005% to about 0.04% of concentration. In some embodiments, the therapeutically effective amount of the composition comprises from about 0.01% to about 0.02% of concentration. In some embodiments, the therapeutically effective amount of the composition comprises about 0.001%, about 0.005%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, or about 50% of concentration. In some embodiments, the therapeutically effective amount of the composition comprises at least about 0.001%, about 0.005%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, or about 50% of concentration. In some embodiments, the therapeutically effective amount of the composition comprises at most about 0.001%, about 0.005%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, or about 50% of concentration. In some embodiments, the therapeutically effective amount of the composition comprises from about 0.001% to about 50%, from about 0.005% to about 45%, from about 0.01% to about 40%, from about 0.015% to about 35%, from about 0.02% to about 30%, from about 0.03% to about 25%, from about 0.04% to about 20%, from about 0.05% to about 15%, from about 0.06% to about 14%, from about 0.07% to about 13%, from about 0.08% to about 12%, from about 0.09% to about 11%, from about 0.1% to about 10%, from about 0.2% to about 9%, from about 0.3% to about 8%, from about 0.4% to about 7%, from about 0.5% to about 6%, from about 0.6% to about 5%, from about 0.7% to about 4%, from about 0.8% to about 3%, from about 0.9% to about 2%, or from about 1% to about 50% of concentration. In some embodiments, the therapeutically effective amount of the composition comprises about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 5.0 mg/day, or 10 mg/day. In some embodiments, the therapeutically effective amount of the composition comprises at least about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 5.0 mg/day, or 10 mg/day. In some embodiments, the therapeutically effective amount of the composition comprises at most about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 5.0 mg/day, or 10 mg/day. In some embodiments, the therapeutically effective amount of the composition ranges from about 0.1 mg/day to about 10 mg/day, from about 0.2 mg/day to about 5.0 mg/day, from about 0.3 mg/day to about 1.0 mg/day, from about 0.4 mg/day to about 0.9 mg/day, from about 0.5 mg/day to about 0.8 mg/day, or from about 0.6 mg/day to about 0.7 mg/day.

The precise therapeutically effective amount is that amount of the composition that will yield the most effective results in terms of efficacy of treatment in a given subject. This amount will vary depending upon a variety of factors, including but not limited to the characteristics of the compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration.

The therapeutically effective amount of the composition disclosed herein can be administered via topical, transdermal, oral, or nasal administration routes. In some embodiments, the topical administration comprises administration of the composition to an affected skin area. Non-limiting examples of dosage forms for topical administration or for transdermal administration of the composition disclosed herein comprise an ointment, cream, suspensions, paste, lotions, powders, solutions, oils, encapsulated gel, liposomes, sprayable aerosol or vapors, solutions, patches, drops, inhalants, or combinations thereof. In some embodiments, sprayable aerosol or vapors additionally comprise customary propellants, for example chlorofluoro-hydrocarbons, and volatile unsubstituted hydrocarbons, for example butane and propane. In other embodiments, transdermal patches may provide an advantage of controlled delivery of the composition disclosed herein to the subject in need thereof. In some embodiments, the dosage forms are made by dissolving, dispersing or otherwise incorporating the composition disclosed herein in a proper medium. In some embodiments, absorption enhancers are also used to increase the flux of the composition across the skin. In some embodiments, the rate of the flux is controlled by either providing a rate-controlling membrane or dispersing the compound in a polymer matrix or gel. In some embodiments, a drug-impregnated solid carrier (e.g., a dressing) can also be used for topical administration and can be non-naturally occurring.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier, diluent or excipient” includes without limitation any adjuvant, carrier, excipient, glidant, sweetening agent, diluent, preservative, dye, colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The section headings used herein are for organizational purposes only and are not to be construed as limiting the subject matter described.

### EXAMPLES

The following examples are included for illustrative purposes only and are not intended to limit the scope of the invention.

### Example 1: Thujopsene Induces Gene Expression of FLG and LOR in Human Primary Keratinocytes

This example demonstrates the induction of gene expression of FLG and LOR in human keratinocytes treated with various concentrations of thujopsene.

Normal human epidermal keratinocytes (NHEKs) were cultured in a conventional way. NHEKs (Life Technologies, Grand Island, NY, USA) were derived from neonatal foreskin and were grown in serum-free EpiLife cell culture medium (Life Technologies) containing 0.06 mmol/L calcium chloride, 1% human keratinocyte growth supplement S7 (Life Technologies), and 1% of gentamicin/amphotericin. To investigate the effects of thujopsene on the expressions of FLG and LOR, NHEKs were seeded at 2×10⁵ cells per well (24-well plate) and differentiated in the presence of 1.3 mmol/L of CaCl₂) for 3 days. Then the cells were stimulated with 0 v/v %, 0.01 v/v % and 0.02 v/v % of thujopsene, IL-4 and IL-13, or IFN-γ for an additional 24 hours. Total RNA was isolated from NHEKs. Gene expression levels of FLG and LOR were determined by real-time RT-PCR using an ABI Prism 7300 sequence detector. Gene expressions of FLG and LOR were normalized to the corresponding 18s RNA transcript in the cultured NHEKs.

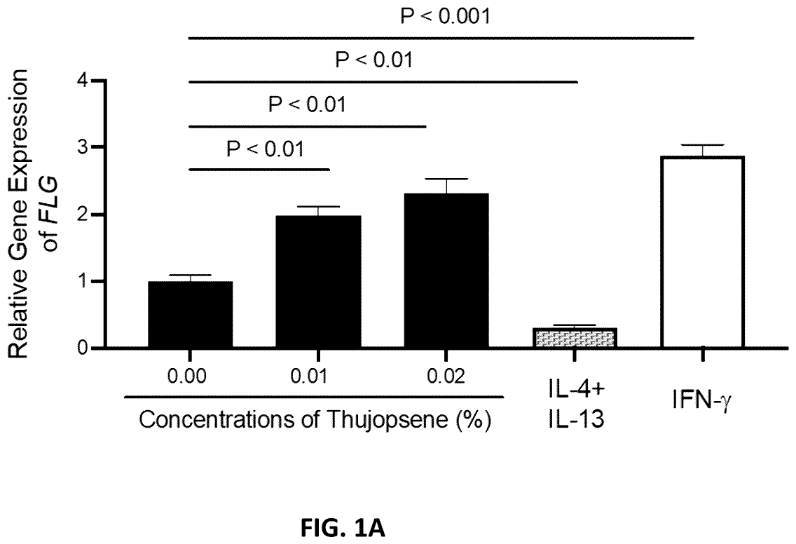

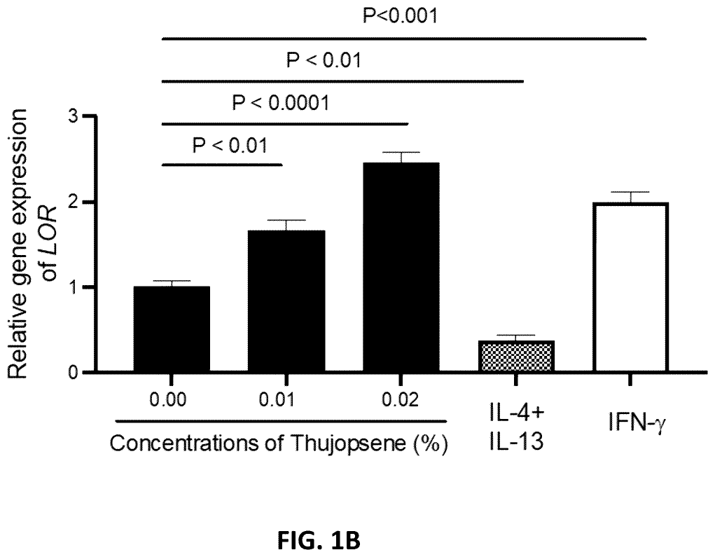

FIGS. 1A and 1B show that thujopsene significantly increases FLG and LOR gene expressions in NHEKs in a dose-dependent manner, respectively.

### Example 2: Thujopsene Attenuates Th2 Cytokine-Mediated Inhibition of FLG in Normal Human Primary Keratinocytes

This example demonstrates that thujopsene overcomes inhibitory effects of Th2 cytokines on FLG. NHEKs were differentiated in the presence of 1.3 mmol/L of CaCl₂) for 3 days. HEKs were incubated with 0.01% of thujopsene for 24 hours, and then the cells were continuously stimulated with IL-4 and IL-13 in the presence or absence of 0.01% of thujopsene for an additional 2 days. The gene expression of FLG was examined using real-time RT-PCR.

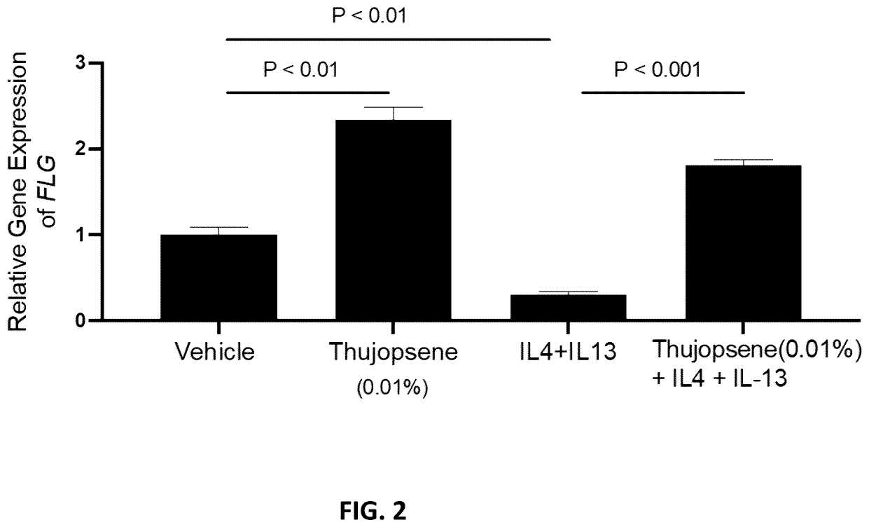

FLG gene expression was significantly (P<0.01) increased in NHEKs treated with a combination of Th2 cytokines and 0.01% of thujopsene compared with NHEKs treated Th2 cytokines alone (FIG. 2).

### Example 3: Thujopsene Increases FLG and LOR in Keratinocytes from Atopic Dermatitis Patients

This example demonstrates treating an inflammatory skin disease or condition, such as atopic dermatitis, with a composition comprising thujopsene as described herein.

Human epidermal keratinocytes taken from atopic dermatitis (AD) patients were differentiated in the presence of 1.3 mmol/L of CaCl₂) for 3 days, and then the cells were stimulated with 0% and 0.01 v/v % of thujopsene for an additional 24 hours. Total RNA was isolated from keratinocytes. Gene expression levels of FLG and LOR were determined by real-time RT-PCR and normalized to the corresponding 18s RNA transcript in the cultured keratinocytes.

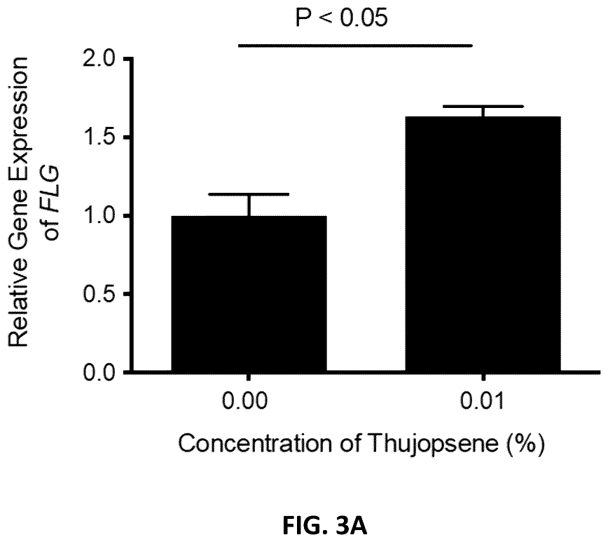

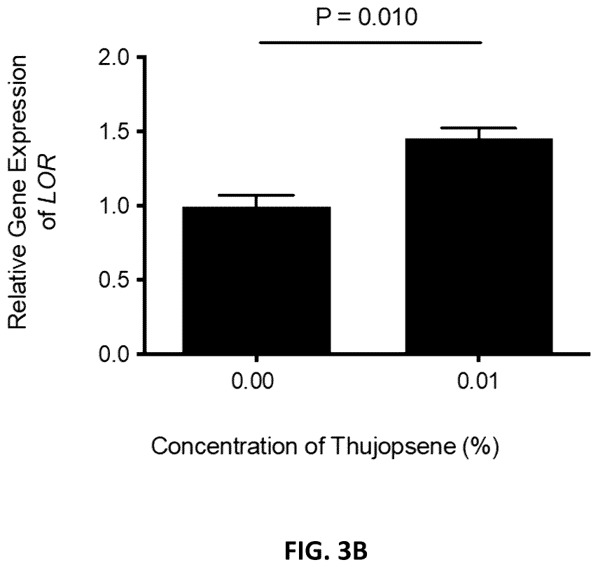

FIGS. 3A and 3B show that thujopsene increases FLG and LOR gene expressions in human primary keratinocytes taken from AD patients, respectively. The gene expressions of FLG (P<0.05) and LOR (P=0.01) were significantly increased in keratinocytes treated with 0.01 v/v % of thujopsene compared to the cells treated with a vehicle lacking thujopsene.

### Example 4: Thujopsene Blocks PM_{2.5}-Mediated Inhibition of FLG and LOR in Normal Human Primary Keratinocytes

To understand the direct relationship between increased PM_{2.5} and epidermal barrier protein production such as FLG and LOR, NHEKs were stimulated with PM_{2.5}, and expressions of FLG and LOR were evaluated.

To investigate the effects of the PM_{2.5} on the expression of FLG and LOR, NHEKs were differentiated in the presence of 1.3 mmol/L of CaCl₂) for 3 days. Then cells were stimulated with PM_{2.5} (1 ng/mL) for an additional 48 hours.

To examine whether thujopsene blocks PM_{2.5}-mediated inhibition of epidermal barrier proteins such as FLG and LOR, NHEKs were incubated with thujopsene (0.01 v/v %) for 24 hours, and then cells were stimulated with PM_{2.5} (1 ng/mL) or combination of thujopsene (0.01 v/v %) and PM_{2.5} for an additional 48 hours. Total RNAs were isolated from the cells, and real time RT-PCR was performed. EpiLife cell culture medium with 0.1% DMSO was used as a vehicle control for all in vitro experiments, as PM_{2.5} was diluted with EpiLife cell culture medium with 0.1% DMSO.

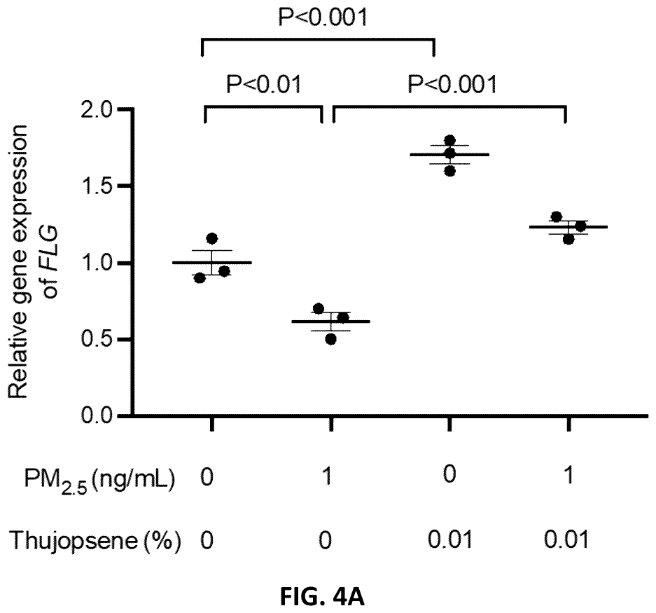

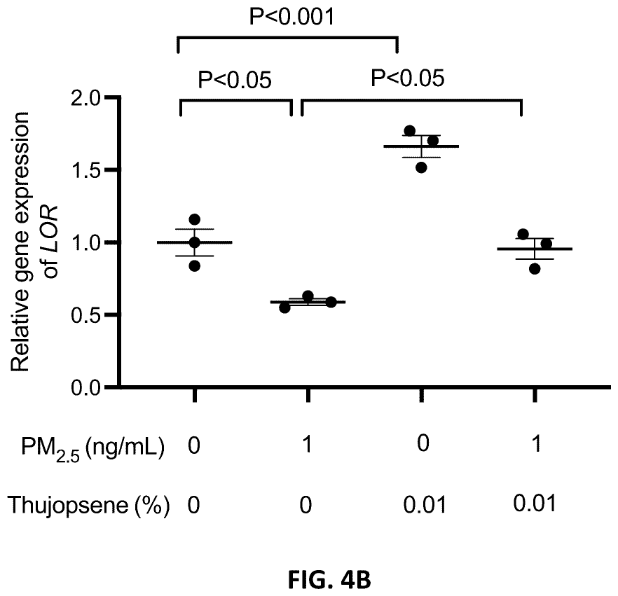

As shown FIGS. 4A and 4B, gene expression of FLG and LOR were significantly (P<0.01) decreased in HEKs treated with PM_{2.5}. However, the decrease of FLG and LOR gene expression were smaller after treatment of 0.01% of thujopsene compared to the case without treatment of thujopsene. Notably, FIG. 4A shows that the level of expression of the FLG gene in NHEK treated with PM_{2.5} and 0.01% of thujopsene was higher than the control. This demonstrates that the composition described herein can completely block the inhibitory effect of PM_{2.5} on the gene expression of the FLG in skin cells. Similarly, in FIG. 4B, the level of expression of the LOR gene in HEK treated with PM_{2.5} and 0.01% of thujopsene was about equal to the control. This demonstrates that the composition described herein can completely block the inhibitory effect of PM_{2.5} on the gene expression of the LOR in skin cells.

### Example 5: Thujopsene Improves Wound Healing and Prevents Skin Cancer Development by Upregulation of HBD-3, LL-37, OR2AT4, and SIRT1

The upregulation of expressions of HBD-3, LL-37, OR2AT4, and/or SIRT1 can promote would healing and the upregulation of SIRT1 can inhibit tumor cell growth in skin cancer. To investigate the effects of thujopsene on expression of RBD-3, LL-37, OR2AT4, and SIRT1, NHEKs were differentiated in the presence of 1.3 mmol/L of CaCl₂) for 3 days. Then cells were stimulated with 0.01% or 0.02% thujopsene for an additional 24 hours. The cell culture medium with 0.1% was used as a vehicle control because thujopsene was diluted with 0.1% of DMSO in cell culture medium.

Total RNA was isolated from to the cells, and real time RT-PCRs were performed by using an ABI Prism 7300 sequence detector.

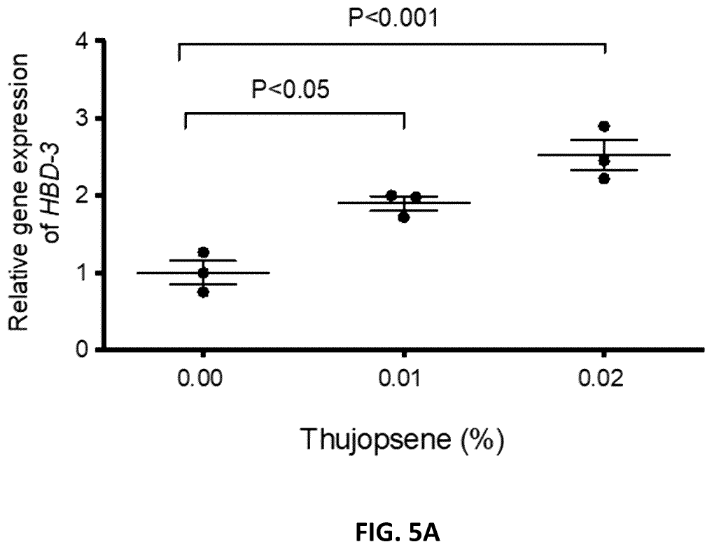

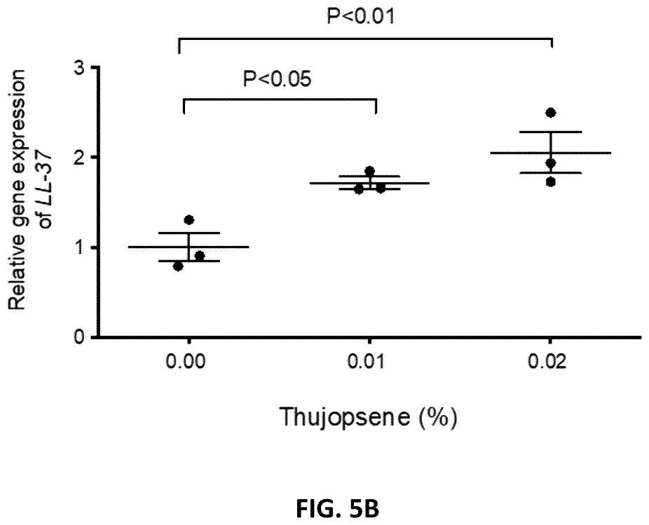

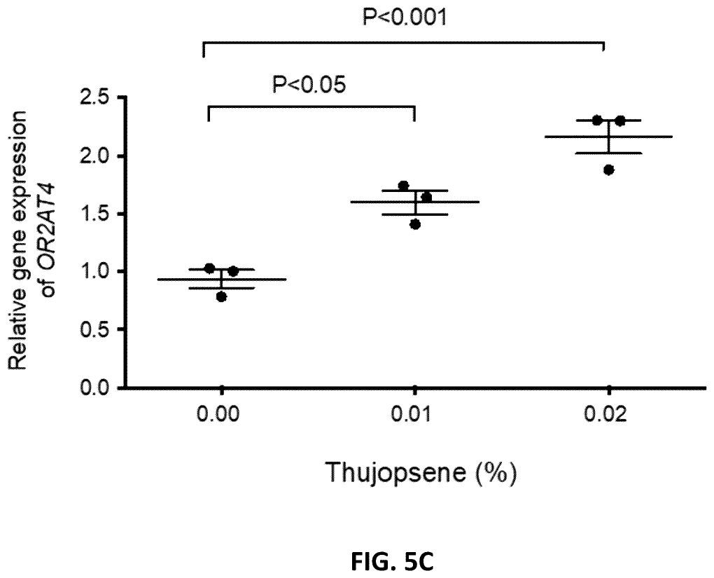

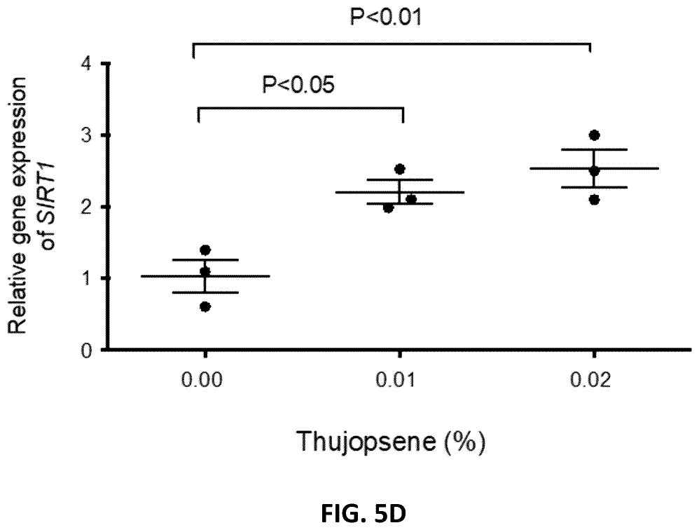

As shown FIG. 5A-5D, gene expression of RBD-3, LL-37, OR2AT4, and SIRT1 were significantly increased in HEKs treated with 0.01% and 0.02% thujopsene as dose-dependent manner, compared to the case without treatment of thujopsene. This example demonstrates that thujopsene can improve wound healing and inhibit skin cancer development via upregulation of gene expressions of RBD-3, LL-37, OR2AT4, and/or SIRT1.

### Example 6: Thujopsene Inhibits Aging and Wrinkling Process

IL-6 aging process and wrinkling and is induced by lipopolysaccharide (LPS) (PMID: 23659624). SIRT1 inhibits skin aging and wrinkling process (PMID: 24011076). To investigate the effects of thujopsene on expression of IL-6 and SIRT1, NHEKs were seeded at 2×10⁵ cells per well and differentiated in the presence of 1.3 mmol/L of CaCl₂) for 3 days. NHEKs were stimulated with 0.01% or 0.02% thujopsene for 24 hours, and then cells were stimulated with LPS (1 μg/mL) alone or a combination of LPS (1 μg/mL) and thujopsene (0.01%) for an additional 24 hours. The cell culture medium with 0.1% DMSO was used as a vehicle. Total RNAs were isolated from cells, and real time RT-PCRs were performed by using an ABI Prism 7300 sequence detector.

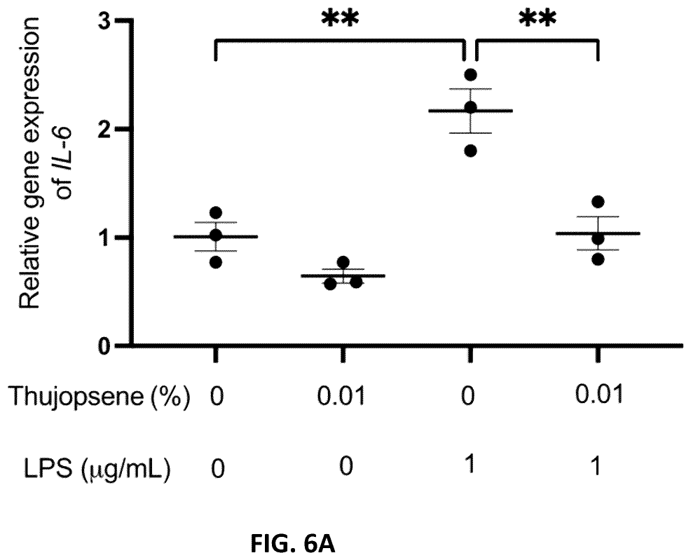

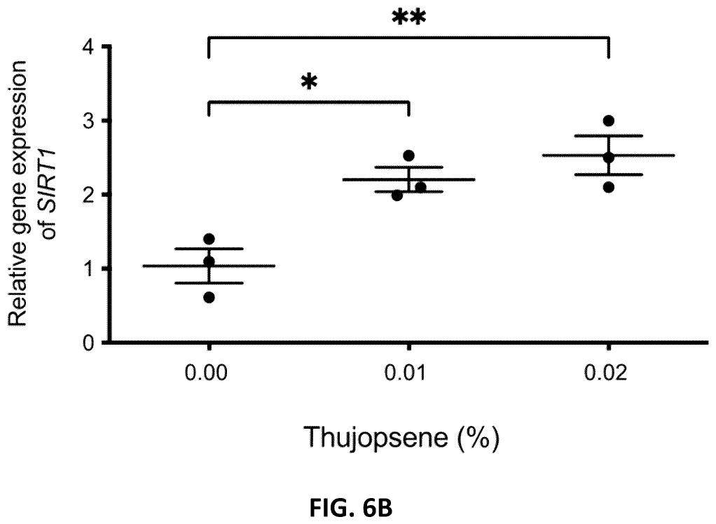

Gene expression of IL-6 was significantly (P<0.01) increased in cells treated with LPS as expected (FIG. 6A), but LPS-induced expression of IL-6 was significantly (P<0.01) attenuated in cells treated with a combination of thujopsene (0.01%) and LPS (1 μg/mL) (FIG. 6A). Gene expression of SIRT1 was significantly increased after treated with 0.01% (P<0.05) and 0.02% (P<0.01) of thujopsene as dose-dependent manner compared to the case with no treatment of thujopsene (FIG. 6B). This example demonstrates that thujopsene provides a beneficial effect on skin aging and wrinkling process by attenuating LPS-induced expression of IL-6 and/or upregulating the expression of SIRT1.

### Example 7: Thujopsene Up to 0.04 v/v % does not Show Cell (Keratinocyte) Toxicity

LDH cell cytotoxicity assay: NHEKs were plated at 2×105 cells per well in a 24-well plate and differentiated in the presence of 1.3 mmol/L of CaCl₂) for 3 days. To examine cell toxicity of thujopsene, the cells were treated with various concentrations of thujopsene for 48 hours, and lactate dehydrogenase (LDH) release was then determined using CytoTox-One Homogeneous Membrane Integrity Assay (Promega, Madison, WI, USA) according to the manufacturer's instructions. Fluorescence was measured on a DTX880 Multimode Detector (Beckman Coulter, Brea, CA, USA) with an excitation wavelength of 560 nm and an emission wavelength of 590 nm. Samples were tested in triplicate and fluorescence results were normalized by subtracting a PBS blank and compared to the keratinocyte LDH release in response to the treatment with 1% triton X-100 solution (maximum LDH release).

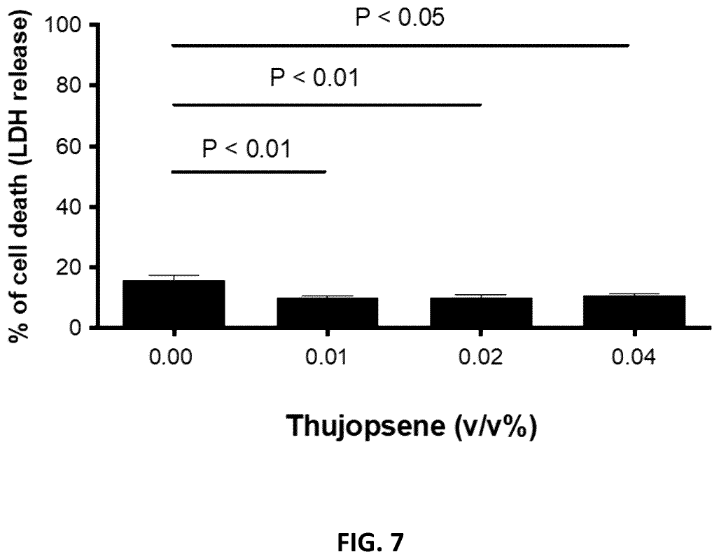

LDH release was significantly decreased in NHEKs treated with thujopsene (up to 4 v/v %) compared to cells treated with media alone (FIG. 7), demonstrating that thujopsene is not toxic to NHEKs, but protects NHEKs.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method for preventing, treating, or ameliorating a skin disease or condition in a subject in need thereof, the method comprising administering to the subject a therapeutically effective amount of a composition comprising at least about 10 v/v % of thujopsene.

Embodiment 2. The method of embodiment 1, wherein the composition comprises at least about 30 v/v % of thujopsene.

Embodiment 3. The method of embodiment 1, wherein the composition comprises at least about 50 v/v % of thujopsene.

Embodiment 4. The method of embodiment 1, wherein the composition comprises at least about 70 v/v % of thujopsene.

Embodiment 5. The method of embodiment 1, wherein the composition comprises at least about 90 v/v % of thujopsene.

Embodiment 6. The method of embodiment 1, wherein the thujopsene has a purity of at least about 80%.

Embodiment 7. The method of embodiment 1, wherein the thujopsene has a purity of at least about 90%.

Embodiment 8. The method of embodiment 1, wherein the thujopsene has a purity of at least about 95%.

Embodiment 9. The method of embodiment 1, wherein the purity of thujopsene is determined by HPLC.

Embodiment 10. The method of embodiment 1, wherein the skin disease or condition comprises a skin inflammatory disease, an allergic disease, a skin wound, a skin aging-related condition, or particulate matter (PM_{2.5})-associated condition.

Embodiment 11. The method of embodiment 10, wherein the skin inflammatory disease comprises atopic dermatitis (AD), contact dermatitis, seborrheic dermatitis, acne, psoriasis (PS), xeroderma, UV-induced inflammation, ichthyosis, or combinations thereof.

Embodiment 12. The method of embodiment 10, wherein the allergic disease comprises anaphylaxis, allergic rhinitis, asthma, atopic dermatitis, food allergies, urticaria, eczema, or combinations thereof.

Embodiment 13. The method of embodiment 10, wherein the skin wound comprises chronic wound, puncture wound, surgical wound, thermal wound, chemical wound, burn, abrasion, avulsion, laceration, or combinations thereof.

Embodiment 14. The method of embodiment 10, wherein the skin aging-related condition comprises wrinkling, sagging, thinning, the appearance of age spots, dryness, or combinations thereof.

Embodiment 15. The method of embodiment 10, wherein the PM_{2.5}-associated condition comprises environmental allergic diseases.

Embodiment 16. The method of embodiment 1, wherein the composition is configured to increase the expression of one or more genes selected from filaggrin (FLG), loricrin (LOR), sirtuin 1 (SIRT1), beta-defensin-3 (RBD-3), cathelicidin (LL-37), and Olfactory Receptor Family 2 Subfamily AT Member 4 (OR2AT4).

Embodiment 17. The method of embodiment 1, wherein the composition is configured to decrease or inhibit the activity of one or more cytokines selected from interleukin (IL)-4, IL-6, IL-13, IL-25, IL-31, and IL-33.

Embodiment 18. The method of embodiment 17, wherein the one or more cytokines is selected from IL-4, IL-6, and IL-13.

Embodiment 19. The method of embodiment 1, wherein the therapeutically effective amount of the composition is administered one to five times a day.

Embodiment 20. The method of embodiment 1, wherein the therapeutically effective amount of the composition is administered two times a day.

Embodiment 21. The method of embodiment 1, wherein the therapeutically effective amount of the composition is administered three times a day.

Embodiment 22. The method of embodiment 1, wherein the therapeutically effective amount of the composition is administered for at least five consecutive days.

Embodiment 23. The method of embodiment 1, wherein the therapeutically effective amount of the composition is administered for at least seven consecutive days.

Embodiment 24. The method of embodiment 1, wherein the therapeutically effective amount of the composition is administered at least for about 15 days, about 21 days, about 24 days, about 28 days, or about 30 days.

Embodiment 25. The method of embodiment 1, wherein the therapeutically effective amount of the composition comprises from about 0.001%, about 0.005%, about 0.010%, about 0.015%, about 0.020%, about 0.025%, about 0.030%, about 0.035%, about 0.040%, about 0.045%, about 0.050%, about 0.055%, about 0.060%, about 0.065%, about 0.070%, about 0.075%, about 0.080%, about 0.085%, about 0.090%, about 0.095%, or about 0.1% of concentration.

Embodiment 26. The method of embodiment 1, wherein the therapeutically effective amount of the composition comprises from about 0.1%, about 0.2%, about 0.3%, about 0.4%, about 0.5%, about 0.6%, about 0.7%, about 0.8%, about 0.9%, or about 1.0% of concentration.

Embodiment 27. The method of embodiment 1, wherein the therapeutically effective amount of the composition comprises from about 1%, about 2%, about 3%, about 4%, about 5%, about 6%, about 7%, about 8%, about 9%, or about 10% of concentration.

Embodiment 28. The method of embodiment 1, wherein the therapeutically effective amount of the composition comprises from about 10%, about 20%, about 30%, about 40%, or about 50% of concentration.

Embodiment 29. The method of embodiment 1, wherein the administering comprises topical administration or transdermal administration.

Embodiment 30. The method of embodiment 29, wherein the topical administration comprises administration of the composition to an affected skin area.

Embodiment 31. The method of embodiment 29, wherein the topical administration comprises administering an ointment, cream, suspensions, paste, lotions, powders, solutions, oils, encapsulated gel, liposomes, sprayable aerosol or vapors, or any combination thereof.

Embodiment 32. A pharmaceutical composition for preventing, treating, or ameliorating a skin disease or condition in a subject in need thereof, the composition comprising at least 10 v/v % of thujopsene.

Embodiment 33. The pharmaceutical composition of embodiment 32, wherein the composition comprises at least about 30 v/v % of thujopsene.

Embodiment 34. The pharmaceutical composition of embodiment 32, wherein the composition comprises at least about 50 v/v % of thujopsene.

Embodiment 35. The pharmaceutical composition of embodiment 32, wherein the composition comprises at least about 70 v/v % of thujopsene.

Embodiment 36. The pharmaceutical composition of embodiment 32, wherein the composition comprises at least about 90 v/v % of thujopsene.

Embodiment 37. The pharmaceutical composition of embodiment 32, wherein the thujopsene has a purity of at least about 80%.

Embodiment 38. The pharmaceutical composition of embodiment 32, wherein the thujopsene has a purity of at least about 90%.

Embodiment 39. The pharmaceutical composition of embodiment 32, wherein the thujopsene has a purity of at least about 95%.

Embodiment 40. The pharmaceutical composition of embodiment 32, wherein the purity of the thujopsene is determined by HPLC.

Embodiment 41. The pharmaceutical composition of embodiment 32, wherein the composition optionally further comprises at least one pharmaceutically acceptable carrier, at least one diluent, at least one excipient or at least one additive.

Embodiment 42. The pharmaceutical composition of embodiment 41, wherein the at least one pharmaceutically acceptable carrier is selected from the group consisting of: dimethyl sulfoxide (DMSO), alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal, and combinations thereof.

Embodiment 43. The pharmaceutical composition of embodiment 41, wherein the at least one diluent comprises buffer, saline, water, DMSO, lactose, or combinations thereof.

Embodiment 44. The pharmaceutical composition of embodiment 41, wherein the at least one excipient is selected from the group consisting of: animal and vegetable fats, oils, waxes, paraffins, starch, tragacanth, cellulose derivatives, polyethylene glycols, silicones, bentonites, silicic acid, talc, zinc oxide, lactose, talc, silicic acid, aluminum hydroxide, calcium silicates, polyamide powder, and combinations thereof.

Embodiment 45. The pharmaceutical composition of embodiment 41, wherein the at least one additive is selected from the group consisting of: a fatty substance, an organic solvent, a solubilizing agent, a thickener, a gelling agent, a softener, an antioxidant, a suspending agent, a stabilizer, a foaming agent, an aromatic, a surfactant, water, an ionic or non-ionic emulsifying agent, a filler, a sequestering agent, a chelating agent, a preservative, vitamins, a blocker, a moisturizing agent, essential oil, a dye, a pigment, a hydrophilic or hydrophobic activator, a lipid vesicle, antiseptics, stabilizing agents, hydrating agents, emulsification promoters or salts and/or buffers for osmotic control, and combinations thereof.

Embodiment 46. The pharmaceutical composition of embodiment 32, wherein the skin disease or condition comprises a skin inflammatory disease, an allergic disease, a skin wound, a skin aging-related condition, or PM_{2.5}-associated condition.

Embodiment 47. The pharmaceutical composition of embodiment 46, wherein the skin inflammatory disease comprises atopic dermatitis (AD), contact dermatitis, seborrheic dermatitis, acne, psoriasis (PS), xeroderma, UV-induced inflammation, ichthyosis, or combinations thereof.

Embodiment 48. The pharmaceutical composition of embodiment 46, wherein the allergic disease comprises anaphylaxis, allergic rhinitis, asthma, atopic dermatitis, food allergies, urticaria, eczema, or combinations thereof.

Embodiment 49. The pharmaceutical composition of embodiment 46, wherein the skin wound comprises chronic wound, puncture wound, surgical wound, thermal wound, chemical wound, burn, abrasion, avulsion, laceration, or combinations thereof.

Embodiment 50. The pharmaceutical composition of embodiment 46, wherein the skin aging-related condition comprises wrinkling, sagging, thinning, the appearance of age spots, dryness, or combinations thereof.

Embodiment 51. The pharmaceutical composition of embodiment 46, wherein the PM_{2.5}-associated condition comprises environmental allergic diseases.

Embodiment 52. The pharmaceutical composition of embodiment 32, wherein the composition is configured to increase the expression of one or more genes selected from FLG, LOR, SIRT1, OR2AT2, and LL-37.

Embodiment 53. The pharmaceutical composition of embodiment 32, wherein the composition is configured to decrease or inhibit the activity of one or more cytokines selected from IL-4, IL-6, IL-13, IL-25, IL-31, and IL-33.

Embodiment 54. The pharmaceutical composition of embodiment 32, wherein the one or more cytokines is selected from IL-4, IL-6, and IL-13.

Embodiment 55. A composition for use in a method for preventing, treating, or ameliorating a skin disease or condition in a subject in need thereof, the method comprising administering to the subject a therapeutically effective amount of a composition comprising at least about 10 v/v % of thujopsene.

Embodiment 56. The composition of embodiment 55, wherein the composition comprises at least about 30 v/v % of thujopsene.

Embodiment 57. The composition of embodiment 55, wherein the composition comprises at least about 50 v/v % of thujopsene.

Embodiment 58. The composition of embodiment 55, wherein the composition comprises at least about 70 v/v % of thujopsene.

Embodiment 59. The composition of embodiment 55, wherein the composition comprises at least about 90 v/v % of thujopsene.

Embodiment 60. The composition of embodiment 55, wherein the thujopsene has a purity of at least about 80%.

Embodiment 61. The composition of embodiment 55, wherein the thujopsene has a purity of at least about 90%.

Embodiment 62. The composition of embodiment 55, wherein the thujopsene has a purity of at least about 95%.

Embodiment 63. The composition of embodiment 55, wherein the purity of thujopsene is determined by HPLC.

Embodiment 64. The composition of embodiment 55, wherein the composition optionally further comprises at least one pharmaceutically acceptable carrier, at least one diluent, at least one excipient or at least one additive.

Embodiment 65. The composition of embodiment 64, wherein the at least one pharmaceutically acceptable carrier is selected from the group consisting of: dimethyl sulfoxide (DMSO), alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal, and combinations thereof.

Embodiment 66. The composition of embodiment 64, wherein the at least one diluent comprises buffer, saline, water, DMSO, lactose, or combinations thereof.

Embodiment 67. The composition of embodiment 64, wherein the at least one excipient is selected from the group consisting of: animal and vegetable fats, oils, waxes, paraffins, starch, tragacanth, cellulose derivatives, polyethylene glycols, silicones, bentonites, silicic acid, talc, zinc oxide, lactose, talc, silicic acid, aluminum hydroxide, calcium silicates, polyamide powder, and combinations thereof.

Embodiment 68. The composition of embodiment 64, wherein the at least one additive is selected from the group consisting of: a fatty substance, an organic solvent, a solubilizing agent, a thickener, a gelling agent, a softener, an antioxidant, a suspending agent, a stabilizer, a foaming agent, an aromatic, a surfactant, water, an ionic or non-ionic emulsifying agent, a filler, a sequestering agent, a chelating agent, a preservative, vitamins, a blocker, a moisturizing agent, essential oil, a dye, a pigment, a hydrophilic or hydrophobic activator, a lipid vesicle, antiseptics, stabilizing agents, hydrating agents, emulsification promoters or salts and/or buffers for osmotic control, and combinations thereof.

Embodiment 69. The composition of embodiment 55, wherein the skin disease or condition comprises a skin inflammatory disease, an allergic disease, a skin wound, a skin aging-related condition, or PM_{2.5}-associated condition.

Embodiment 70. The composition of embodiment 69, wherein the skin inflammatory disease comprises atopic dermatitis (AD), contact dermatitis, seborrheic dermatitis, acne, psoriasis (PS), xeroderma, UV-induced inflammation, ichthyosis, or combinations thereof.

Embodiment 71. The composition of embodiment 69, wherein the allergic disease comprises anaphylaxis, allergic rhinitis, asthma, atopic dermatitis, food allergies, urticaria, eczema, or combinations thereof.

Embodiment 72. The composition of embodiment 69, wherein the skin wound comprises chronic wound, puncture wound, surgical wound, thermal wound, chemical wound, burn, abrasion, avulsion, laceration, or combinations thereof.

Embodiment 73. The composition of embodiment 69, wherein the skin aging-related condition comprises wrinkling, sagging, thinning, the appearance of age spots, dryness, or combinations thereof.

Embodiment 74. The composition of embodiment 69, wherein the PM_{2.5}-associated condition comprises environmental allergic diseases.

Embodiment 75. The composition of embodiment 55, wherein the composition is configured to increase the expression of one or more genes selected from FLG, LOR, SIRT1, RBD-3, OR2T2, and LL-37.

Embodiment 76. The composition of embodiment 55, wherein the composition is configured to decrease or inhibit the activity of one or more cytokines selected from IL-4, IL-6, IL-13, IL-25, IL-31, and IL-33.

Embodiment 77. The composition of embodiment 76, wherein the one or more cytokines is selected from IL-4, IL-6, and IL-13.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for preventing, treating, or ameliorating a skin disease or condition in a subject in need thereof, the method comprising administering to the subject a therapeutically effective amount of a composition comprising at least about 10 v/v % of thujopsene.

2. The method of claim 1, wherein the composition comprises at least about 30, 50, 70, or 90 v/v % of thujopsene.

3. (canceled)

4. (canceled)

5. (canceled)

6. The method of claim 1, wherein the thujopsene has a purity of at least about 80, 90, or 95%.

7. (canceled)

8. (canceled)

9. (canceled)

10. The method of claim 1, wherein the skin disease or condition comprises a skin inflammatory disease, an allergic disease, a skin wound, a skin aging-related condition, or a particulate matter (PM_{2.5})-associated condition.

11. The method of claim 10, wherein the skin inflammatory disease comprises atopic dermatitis (AD), contact dermatitis, seborrheic dermatitis, acne, psoriasis (PS), xeroderma, UV-induced inflammation, ichthyosis, or combinations thereof.

12. The method of claim 10, wherein the allergic disease comprises anaphylaxis, allergic rhinitis, asthma, atopic dermatitis, food allergies, urticaria, eczema, or combinations thereof.

13. The method of claim 10, wherein the skin wound comprises chronic wound, puncture wound, surgical wound, thermal wound, chemical wound, burn, abrasion, avulsion, laceration, or combinations thereof.

14. The method of claim 10, wherein the skin aging-related condition comprises wrinkling, sagging, thinning, appearance of age spots, dryness, or combinations thereof.

15. The method of claim 10, wherein the PM_{2.5}-associated condition comprises environmental allergic diseases.

16. The method of claim 1, wherein the composition is configured to increase expression of one or more genes selected from filaggrin (FLG), loricrin (LOR), sirtuin 1 (SIRT1), beta-defensin-3 (HBD-3), cathelicidin (LL-37), and Olfactory Receptor Family 2 Subfamily AT Member 4 (OR2AT4).

17. The method of claim 1, wherein the composition is configured to decrease or inhibit an activity of one or more cytokines selected from interleukin (IL)-4, IL-6, IL-13, IL-25, IL-31, and IL-33.

18. (canceled)

19. The method of claim 1, wherein the therapeutically effective amount of the composition is administered one to five times a day.

20. (canceled)

21. (canceled)

22. The method of claim 1, wherein the therapeutically effective amount of the composition is administered for at least about 5 days, about 7 days, about 15 days, about 21 days, about 24 days, about 28 days, or about 30 days.

23. (canceled)

24. (canceled)

25. The method of claim 1, wherein the therapeutically effective amount of the composition comprises about 0.001%, about 0.01%, about 0.05%, about 0.1%, about 0.2%, about 0.3%, about 0.4%, about 0.5%, about 0.6%, about 0.7%, about 0.8%, about 0.9%, or about 1.0% of concentration.

26. (canceled)

27. (canceled)

28. (canceled)

29. (canceled)

30. (canceled)

31. (canceled)

32. A composition for preventing, treating, or ameliorating a skin disease or condition in a subject in need thereof, the composition comprising at least about 10 v/v % of thujopsene.

33. (canceled)

34. (canceled)

35. (canceled)

36. (canceled)

37. (canceled)

38. (canceled)

39. (canceled)

40. (canceled)

41. The composition of claim 32, wherein the composition further comprises at least one pharmaceutically acceptable carrier, at least one diluent, at least one excipient or at least one additive.

42. The composition of claim 41, wherein the at least one pharmaceutically acceptable carrier is selected from the group consisting of: dimethyl sulfoxide (DMSO), alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetate, borneol, bornyl acetate, alpha-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal, and combinations thereof.

43. The composition of claim 41, wherein the at least one diluent comprises buffer, saline, water, DMSO, lactose, or combinations thereof.

44. The composition of claim 41, wherein the at least one excipient is selected from the group consisting of: animal and vegetable fats, oils, waxes, paraffins, starch, tragacanth, cellulose derivatives, polyethylene glycols, silicones, bentonites, silicic acid, talc, zinc oxide, lactose, talc, silicic acid, aluminum hydroxide, calcium silicates, polyamide powder, and combinations thereof.

45. The composition of claim 41, wherein the at least one additive is selected from the group consisting of: a fatty substance, an organic solvent, a solubilizing agent, a thickener, a gelling agent, a softener, an antioxidant, a suspending agent, a stabilizer, a foaming agent, an aromatic, a surfactant, water, an ionic or non-ionic emulsifying agent, a filler, a sequestering agent, a chelating agent, a preservative, vitamins, a blocker, a moisturizing agent, essential oil, a dye, a pigment, a hydrophilic or hydrophobic activator, a lipid vesicle, antiseptics, stabilizing agents, hydrating agents, emulsification promoters or salts and/or buffers for osmotic control, and combinations thereof.

46.-77. (canceled)
